(19) United States
(12) Patent Application Publication
(10) Pub. No.: US 2016/0207847 A1
(43) Pub. Date: Jul. 21, 2016

(54) PRODUCTION OF BUTADIENE FROM ETHENE

(71) Applicant: EVONIK DEGUSSA GMBH, Essen (DE)

(72) Inventors: Guido STOCHNIOL, Haltern am See (DE); Felix GÄRTNER, Haltern am See (DE)

(73) Assignee: EVONIK DEGUSSA GMBH, Essen (DE)

(21) Appl. No.: 14/996,495

(22) Filed: Jan. 15, 2016

(30) Foreign Application Priority Data
Jan. 19, 2015 (DE) 102015200702.2

(51) Int. Cl. C07C 2/08; C07C 5/48; C07C 5/25

(52) U.S. Cl. CPC C07C 2/08; C07C 5/2506; C07C 5/48

(57) ABSTRACT

The invention relates to a process for the production of 1,3-butadiene from ethene by way of oligomerization and oxidative dehydrogenation.

It is based on the finding that the 1-butene content of a feed material of an oxidative dehydrogenation must not be too high and must therefore be reduced. This occurs by way of isomerization which, however, does not take place from 2-butene to 1-butene, but vice versa from 1-butene to 2-butene. If, according to the invention, isomerization is carried out between the ethene dimerization and the oxidative dehydrogenation of 1-butene to 2-butene, it is possible to achieve a high yield of butadiene and reduce the formation of by-product.

The invention relates to a process for the production of 1,3-butadiene from ethene by way of oligomerization and oxidative dehydrogenation.

1,3-Butadiene (CAS no. 106-99-0) is an important commodity chemical in the chemical industry, It is the starting component in important polymers having various possible uses, including the sector of the automotive industry.

As well as 1,3-butadiene, 1,2-butadiene also exists, but the latter is of little interest because of its low industrial significance. Wherever “butadiene” or “BD” is discussed for short, then 1,3-butadiene is always intended.

A general introduction to the chemical and physical properties of butadiene and production thereof can be found in:

Grub, J. and Laser, E. 2011. Butadiene [Butadienes]. Ullmann's Encyclopedia of Industrial Chemistry.

Butadiene is currently obtained on an industrial scale mostly by extractive removal from C4 streams. C4 streams are mixtures of different hydrocarbons having four carbon atoms which are produced in mineral oil crackers as coproduct in the production of ethylene and propylene. In the future, a worldwide increasing demand for butadiene will face a shortage of butadiene-containing C4 streams. The reason is an altered raw materials situation and restructuring of refinery processes.

An alternative method for controlled and coproduct-free production of butadiene is the oxidative dehydrogenation (ODH) of n-butene.

The butenes are the four isomeric substances 1-butene, cis-2-butene, trans-2-butene and isobutene. 1-Butene and the two 2-butenes belong to the group of the linear butenes, while isobutene is a branched olefin. The linear C4-olefins 1-butene, cis-2-butene and trans-2-butene are also summarized as “n-butene”.

A current overview of the chemical and physical properties of the butenes and their industrial work-up and utilization is provided by:

F. Geilen, G. Stochniol, S. Peitz and E. Schulte-Koeme: Butenes. Ullmann's Encyclopedia of Industrial Chemistry. (2013)

Just like butadiene, butenes are obtained in the cracking of mineral oil fractions in a steamcracker or in a fluid catalytic cracker (FCC). However, the butenes are not obtained in pure form but as a so-called “C4 cut”. This is a mixture of hydrocarbons having four carbon atoms that has a different composition depending on the origin and which also comprises saturated C4 hydrocarbons (alkanes) alongside C4-olefins. In addition, traces of hydrocarbons having more or fewer than four carbon atoms (for example, but not exclusively, propane and/or pentenes) and other organic or inorganic accompanying substances may be present. Alternative sources of butenes are, for example, chemical processes such as the dehydrogenation of butane, ethylene dimerization, metathesis, methanol-to-olefin technology, Fischer-Tropsch, and the fermentative or pyrolytic conversion of renewable raw materials.

Since butadiene-containing C4 streams will be in short supply in future, there is presently increased research into the production of butadiene by way of oxidative dehydrogenation from butenes.

Jung et al, Catal, Surv. Asia 2009, 13, 78-93 describe a large number of transition metal mixed oxides, in particular ferrites or bismuth molybdates which are suitable as heterogeneous catalysts for the ODH.

US2012130137A1 also describes a bismuth molybdate over which butene-containing streams can be oxidatively dehydrogenated with an oxygenous gas to give butadiene.

In order to optimally utilize available raw material sources, there have been descriptions of processes in which the oxidative dehydrogenation of butane to butadiene is used together with other reactions in a multistage process concept.

For example, WO2006076025 or WO2004007408A1 describes a process which couples an autothermally catalysed, nonoxidative dehydrogenation of butane to butene with an oxidative dehydrogenation of the butenes obtained to give butadiene. This opens up a direct route for the production of butadiene from butane, which is little utilized industrially in chemical conversions except for the production of maleic anhydride. The disadvantage of this process is large recycling streams as a result of recycling butane, which increase the apparatus and operating costs, By means of the process described in US20110040134, it is also possible to use isobutene-containing streams for the oxidative dehydrogenation of butane to butadiene. This is enabled by a skeletal isomerization of isobutene to 2-butene that precedes the oxidative dehydrogenation. The disadvantage of this process is that it is based on isobutene, a raw material that can be used in other ways with a greater addition of value. The production of butadiene from isobutene is therefore uneconomic.

The butene isomers 1-butene and 2-butene can be converted over different catalysts at different rates to give butadiene (WO2009119975). By layering a double fixed catalyst bed, the overall yield can be improved significantly compared to a comparative experiment with only one catalyst type. In the examples mentioned, ferrite and mixed bismuth/molybdenum oxide catalysts are used.

However, different optimal operating conditions of the catalysts lead to different industrial lifetimes of individual catalysts, which entails comparatively frequent interruption of operation for exchange of the individual catalysts.

U.S. Pat. No. 3,479,415 describes a process in which 2-butene-containing streams are converted via an isomerization and subsequent separation step to 1-butene. The distillatively enriched 1-butene is subsequently converted in an oxidative dehydrogenation stage to butadiene. A disadvantage is the additional energy-intensive separation step for production of enriched 1-butene. Moreover, 1-butane is a raw material having a comparable addition of value potential to 1,3-butadiene, and so the processing of 1-butene to butadiene makes barely any sense in economic terms.

Already of more economic interest is the production of butadiene from n-butene mixtures which also comprise a high fraction of 2-butene as well as 1-butene.

A process for direct utilization of such streams in butadiene production is described in EP2256101A2, The oxidative dehydrogenation of the n-butene present in the input stream is effected in a double fixed bed comprising two different catalyst systems. The first catalyst is a bismuth molybdate over which the 1-butene present in the butane mixture is converted to butadiene. The conversion of the 2-butene is catalysed using a zinc-ferrite system. It is an undisputed advantage of this process that it allows the direct utilization of feed mixtures which comprise not only 1-butene but also 2-butene. It is a disadvantage of this process that the two 2-butenes are less reactive compared to 1-butene, and therefore the residence time of the n-butenes in the double fixed bed is unnecessarily long: here, the slower reaction determines the process duration. The higher the proportion of 2-butene compared to 1-butene, the greater the adverse effect. Therefore, the process is tied to a restricted 1-butene to 2-butene ratio, in order to achieve sufficiently high n-butene conversions. If variable raw material sources afford butene mixtures having a variable ratio of 1-butene to 2-butene, losses in the butadiene yield have to be accepted in this process.

A disadvantage of this process is that it is assigned to the naptha-based raw material n-butene.

Since the recovery of mineral oil is in decline, C4-containing raw material mixtures are in short supply and are becoming expensive.

As a result of the increasing recovery of unconventional natural gas, C2-based raw materials are currently more cost-effective. Natural gas comprises ethane, which can be converted by dehydrogenation to the C2-olefin ethene. Ethene can in turn be dimerized using established technology to give butenes which can then be further reacted to give butadiene by oxidative dehydrogenation.

This route is fundamentally indicated in the German Patent Application 102013226370, which was unpublished at the filing date of the application, in which an n-butene obtainable by ethylene dimerization is firstly subjected to an isomerization of 2-butene to 1-butene and then the isomerizate is subjected to an oxidative dehydrogenation to give butadiene.

Investigations reveal that during the dimerization of ethene, predominantly 1-butene is formed, meaning that the oligomerizate is rich in 1-butene. If the 2-butenes present in the oligomerizate are further isomerized in the direction 1-butene, en isomerizate is obtained which is very rich in 1-butene.

Surprisingly, the oxidative dehydrogenation of streams which have a very high content o-butane leads to unexpected problems:

It has been observed that the very exothermic conversion of 1-butene to butadiene causes very high temperature peaks which lead to nonselective superoxidation reactions as well as increased formation of secondary reactions, meaning that the yield of butadiene drops. This effect can be observed during the oxidative dehydrogenation of feed materials which comprise more than 90% by weight of 1-butene and increases with an increase in 1-butene content, meaning that it is most marked during the oxidative dehydrogenation of pure 1-butene.

Against this background, the route indicated in DE 102013226370 of ethylene dimerization, isomerization of the oligomerizate in the direction 1-butene and oxidative dehydrogenation of the isomerizate is not suited to producing butadiene from ethene in an economical manner: This is because the isomerizate introduced into the oxidative dehydrogenation during this procedure is already so rich in 1-butene that the butadiene yield drops further and the by-product formation increases.

The object of the invention is therefore to indicate a process for producing butadiene from ethene which achieves a high butadiene yield and a low by-product formation.

This object is achieved by a process having the following steps:

a) providing a feed material which comprises ethene or consists of ethene;

b) subjecting the feed material to an oligomerization to give an oligomerizate, where the oligomerizate comprises at least 1-butene;

c) optionally working-up of the oligomerizate to give a fraction rich in 1-butene;

d) subjecting the oligomerizate or the fraction rich in 1-butene to an isomerization to give an isomerizate, where the content of 2-butene in the isomerizate is greater than the content of 2-butene in the oligomerizate or the fraction rich in 1-butene;

e) subjecting the isomerizate to an oxidative dehydrogenation to give a product mixture comprising butadiene.

The invention is based on the finding that the 1-butene content of the feed material of the oxidative dehydrogenation should not be too high and must therefore be reduced. This occurs by way of an isomerization which, however, does not take place from 2-butene to 1-butene, but vice versa from 1-butene to 2-butene, In this way, the content of 2-butene increases as a result of the isomerization, meaning that the 2-butene content of the isomerizate is greater than the substance mixture introduced into the isomerization, i.e. the oligomerizate or the fraction rich in 1-butene separated off from the oligomerizate. The 1-butene content therefore decreases during the isomerization, meaning that an isomerizate is obtained which does not cause the described problems in the oxidative dehydrogenation.

Only if, in accordance with the invention, isomerization is carried out between the ethene dimerization and the oxidative dehydrogenation of 1-butene to 2-butene can a high yield of butadiene be achieved and the by-product formation be reduced.

This finding is surprising in so far as in the past it was always attempted to supply the oxidative dehydrogenation with a feed mixture that was as rich as possible in 1-butene. The present invention deviates from this.

Preferably, the isomerizate which is passed to the oxidative dehydrogenation should comprise less than 90% by weight of 1-butene. Ideally, the isomerizate obeys the following specification, which adds up to 100% by weight:
1-Butene: 20% by weight to 90% by weight
cis-2-Butene 5% by weight to 40% by weight
trans-2-Butene 5% by weight to 40% by weight
isobutene 0% by weight to 1% by weight
Other substances: 0% by weight to 1% by weight

In order to obtain such an isomerizate, an isomerization of 1-butene to 2-butene is carried out under the following conditions:

Temperature of 400° C. to 550° C., pressure 8*10⁵ Pa to 12*10⁵ Pa. A heterogeneous catalyst based on magnesium fluoride can be used. A corresponding isomerization is described in U.S. Pat. No. 2,377,352 A.

Useful isomerization catalysts are in principle all catalysts which catalyse the double bond isomerization of 1-butene to 2-butene. In general, these are mixed oxide compositions comprising aluminium oxides, silicon oxides, and mixtures and mixed compounds thereof, zeolites and modified zeolites, aluminas, hydrotalcites, borosilicates, alkali metal oxides or alkaline earth metal oxides, and mixtures and mixed compounds of the components mentioned. The catalytically active materials mentioned may additionally be modified by oxides of the elements Mg, Ca, Sr, Na, Li, K, Ba, La, Zr, Sc, and oxides of the manganese group, iron group and cobalt group. The metal oxide content, based on the overall catalyst, is 0.1% to 40% by weight, preferably 0.5% to 25% by weight.

Suitable isomerization catalysts are described, inter alia, in DE3319171, DE3319099, US4289919, U.S. Pat. No. 3,479,415, EP234498, EP129899, U.S. Pat. No. 3,475,511, U.S. Pat. No. 4,749,819, U.S. Pat. No. 4,992,613, U.S. Pat. No. 4,499,326, U.S. Pat. No. 4,217,244, WO03076371 and WO02096843.

In a particularly preferred form, the isomerization catalyst comprises at least two different components, the two components having been mixed with one another or the first component having been applied to the second component. In the latter case, the catalyst is frequently of the type known as a supported catalyst, in which the first component constitutes the essentially catalytically active substance, while the second component functions as support material. However, some catalysis experts express the view that the support of a conventional supported catalyst is likewise catalytically active. For this reason, reference is made in this context, without any consideration of any catalytic activity, to a first component and a second component.

Very particularly suitable isomerization catalysts have proven to be two-component systems which comprise an alkaline earth metal oxide on an acidic aluminium oxide support or on a mixture of Al2O3 and SiO2. The alkaline earth metal oxide content, based on the overall catalyst, is 0.5% to 30% by weight, preferably 0.5% to 20% by weight. Alkaline earth metal oxides used may be magnesium oxide and/or calcium oxide and/or strontium oxide and/or barium oxide.

The second component (i.e. as “support”) used is aluminium oxide or silicon dioxide or a mixture of aluminium oxide and silicon dioxide or an alumosilicate.

A catalyst which is based on MgO and aluminosilicate and is suitable for isomerization is described in EP1894621B1.

A system of even better suitability as isomerization catalyst is that known from EP0718036A1, in which strontium oxide as first component has been applied to aluminium oxide as second component. The strontium content here is between 0.5% and 20% by weight, based on the total catalyst weight. Alternatively, it is possible to use a heterogeneous catalyst in which magnesium oxide as first component has been mixed with an aluminosilicate as second component. Catalysts of this kind are disclosed in EP1894621A1.

The isomerization is supplied with a feed mixture which is rich in 1-butene. It should obey the following specification which adds up to 100% by weight:
1-Butene: 80% by weight to 100% by weight
cis-2-Butene 0% by weight to 10% by weight
trans-2-Butene 0% by weight to 10% by weight
Isobutene 0% by weight to 1% by weight
Other substances: 0% by weight to 1% by weight

It is evident from the comparison of the two specifications that the content of 1-butene decreases greatly as a result of the isomerization, whereas the content of 2-butene accordingly increases considerably. The thermodynamic equilibrium between 1-butene and 2-butene is shifted significantly in the direction of the two 2-butenes; the 1-butene is quasi converted to 2-butene.

The feed mixture for the isomerization is either the oligomerizate obtained from the ethene dimerization or a fraction rich in 1-butene which has been separated off from this oligomerizate.

According to the invention, the working-up of the oligomerizate obtained from the ethene oligomerization is namely only optional: It is possible to convey the oligomerizate to the isomerization without work-up, i.e. separation off of the components present therein. Whether this is possible depends on the composition of the oligomerizate. If it already satisfies the aforementioned specification, it does not require work-up and the oligomerizate is conveyed as a whole to the isomerization.

In many cases, the ethene dimerization, however, will also produce higher oligomers, for example C6 and C8-olefins. In the interest of by-product formation, it is necessary to avoid conveying higher olefins to the oxidative dehydrogenation. Consequently, firstly a fraction rich in 1-butene is separated off from the oligeromerizate corresponding to the above specification of the feed mixture of the isomerization which comprises for example ail of the C4-olefins present in the oligomerizate.

This C4 fraction is then isomerized, the other oligomers are removed. The advantage of the interim separating off of the fraction rich in 1-butene is that isomerization and oxidative dehydrogenation are not unnecessarily burdened with substances that are not to be reacted and the by-product formation drops.

The C4-fraction of the oligomerizate comprising 1-butene and 2-butene also does not require to be separated further since a mixture of 1-butene and 2-butene can be conveyed to the oxidative dehydrogenation.

Fully developed technology can be used for the ethylene oligomerization. Mention is to be made for example of the alphabutol process from IFP/Axens or the Shell Higher Olefins Process (SHOP).

Also of suitability are the alpha-SABLIN® process from Linde and SABIC, and also the ethylene oligomerization from ChevronPhillips and INEOS.

A principle distinction to be made in this connection is that between heterogeneously catalysed and homogeneously catalysed oligomerizations. Examples of homogeneously catalysed examples in the patent literature are WO2005/123633, US2013/0086128 A1. A disadvantage of the homogeneously catalysed processes is that the catalyst is present in the same phase as the reaction mixture and therefore has to be separated off from the oligomerizate, entailing costs. The ethene dimerization is therefore preferably carried out over a heterogeneous catalyst.

This is because the problem of catalyst removal does not exist for heterogeneously catalysed processes in which the catalyst is present as solid and remains in the reactor. Ethylene oligomerization over a solid Si/Al/Ni system is described in US863772262. However, this process takes place in the gas phase, which is disadvantageous for the utilization of space in the reactors.

Moreover, the established process steps of the further processing of butenes and octenes in the liquid phase take place such that this gas-phase process is not directly compatible with existing technology. A necessary liquefaction of the butenes and octenes obtained in the gas phase requires additional energy.

The gas-phase process shown in WO2010/117539A1 for the oligomerization of ethylene diluted in a FCC gas over a zeolitic Ni catalyst can also not be incorporated directly into an established production run for C₄/C₈ utilization.

The same is also true for the heterogeneous gas-phase oligomerization of ethene over a nickel-containing zeolite described in U.S. Pat. No. 4,717,782, The feed mixture can also comprise C₄-paraffins and inert gases. U.S. Pat. No. 8,837,722 also describes the oligomerization of ethene in the gas phase over a heterogeneous catalyst of Ni/Al on a support made of Al₂O₃/SiO₂. Inert gases such as nitrogen, argon or helium may be present.

A mixed form between heterogeneous and homogeneous C₂-oligomerization is shown in US2013/0158321A1. Here, ethene is firstly dimerized homogeneously to butenes and these are then converted to octenes by heterogeneous catalysis over a solid nickel catalyst. Both reaction stages take place in the liquid phase in the presence of hexane. The reaction discharge from the first stage has to be neutralized with base and freed from the homogeneous catalyst (triethylaluminium) by distillation. In industrial practice, this is very complex.

U.S. Pat. No. 2,581,228 describes the heterogeneously catalysed oligomerization of ethene in the presence of an inert solvent. The solvent should be a relatively high-boiling, inert material, preferably a relatively high-boiling alkene or cycloalkene. The catalyst used is a nickel/aluminium system on silica gel. The reaction mixture is a slurry from which the gel-like catalyst can be recovered. Corresponding expenditure on apparatus will be entailed for this.

Particularly preferably, the ethene dimerization to the oligomerizate takes place as follows:
Temperature: 40° C. to 180° C.
Pressure: 2 to 10 MPa
Ethene concentration: 2% by weight to 30% by weight
Other substances: 70% by weight to 98% by weight
Weight hourly space velocity, (g(ethylene)/g(catalyst mass)/h): 0.1 to 5.0

In this context, the temperature means the temperature which is established in the reactor apparatus. The actual reaction temperature may differ therefrom.

The concentration of ethene and the other substances of course add up to 100% by weight.

The feed material used for the ethylene dimerization is either pure ethene or a mixture comprising ethene.

Preferably, the feed material for the ethylene dimerization obeys the following specification which adds up to 100% by weight:
Ethene: 5% by weight to 60% by weight
Other substances: 50% to 95% by weight

The other substances may be solvents which behave in an inert or noninert manner in the oligomerization. The other substances may be alkenes and alkanes in each case having three to eight carbon atoms or mixtures thereof.

Finally, still with regard to the oxidative dehydrogenation:

The reaction conditions for oxidative dehydrogenation are preferably at the following values:
temperature: 250° C. to 500° C., especially 300° C. to 420° C.
Pressure: 0.08 to 1.1 MPa, especially 0.1 to 0.8 MPa
weight hourly space velocity (g(butenes)/g(active catalyst composition)/h): 0.1 h−1 to 6.0 h−1, in particular 0.15 h−1 to 3.0 h−1

In this context, the temperature means the temperature which is established in the reactor apparatus. The actual reaction temperature may differ therefrom. However, the reaction temperature, i.e. the temperature measured at the catalyst, will likewise fluctuate within the ranges specified.

Catalysts used for the oxidative dehydrogenation may in principle be all the catalysts suitable for the oxidative dehydrogenation of n-butene to butadiene. Two catalyst classes in particular are useful for this purpose, namely mixed metal oxides from the group of the (modified) bismuth molybdates, and also mixed metal oxides from the group of the (modified) ferrites.

Very particular preference is given to using catalysts from the group of bismuth molybdates.

Bismuth molybdates are understood to mean catalysts of formula (I)

(Moe Bib Fec (Co+Ni)d De Ef Fg Gh Hi) Ox (I)

with the meanings

D; at least one of the elements from W, P,

E: at least one of the elements from Li, K, Na, Rb, Cs, Mg, Ca, Ba, Sr,

F: at least one of the elements from Cr, Ce, Mn, V,

G: at least one of the elements from Nb, Se, Te, Sm, Gd, La, Y, Pd, Pt, Ru, Ag, Au,

H: at least one of the elements from Si, Al, Ti, Zr and

the coefficients a to i represent rational numbers selected the following ranges, including the specified limits:

a=10 to 12

b=0 to 5

c=0.5 to 5

d=2 to 15

e=0 to 5

f=0.001 to 2

g=0 to 5

h=0 to 1.5

i=0 to 800

and

x is a number which is determined by the valency and frequency of the elements other than oxygen.

Catalysts of this kind are obtained, for example, by the production steps of co-precipitation, spray drying and calcination. The powder obtained in this way can be subjected to a shaping operation, for example by tableting, extrusion or coating of a support. Catalysts of this kind are described in U.S. Pat. No. 8,003,840, U.S. Pat. No. 8,008,227, US 2011034328 and in U.S. Pat. No. 7,579,501.

The Bi—Mo catalyst in whose presence the oxidative dehydrogenation is carried out is consequently a heterogeneous catalyst. It is preferably present in powder form, i.e. not in the otherwise customary macroscopic form of granules, rings, pellets, tablets and so on.

The butadiene to be produced is in a product mixture which results from the oxidative dehydrogenation. The product mixture comprises, as well as the butadiene target product, unconverted constituents of the butene mixture and unwanted by-products of the oxidative dehydrogenation. More particularly, the product mixture comprises, according to the reaction conditions and composition of the butene mixture provided, butane, nitrogen, residues of oxygen, carbon monoxide, carbon dioxide, water (steam) and unconverted butene. In addition, the product mixture may contain traces of saturated and unsaturated hydrocarbons, aldehydes and acids. In order to separate off the desired butadiene from these undesired accompanying components, the product mixture is subjected to a butadiene separation, in the course of which 1,3-butadiene is isolated from the other constituents of the product mixture.

For this purpose, the product mixture is preferably first cooled and quenched with water in a quench column. With the aqueous phase thus obtained, water-soluble acids and aldehydes, and also high boilers, are removed. The product mixture thus prepurified, after a possible compression, passes into an absorption/desorption step or into a membrane process for removal of the hydrocarbons having four carbon atoms present therein. The butadiene can be obtained from this desorbed C4-hydrocarbon stream for example by extractive distillation.

The butadiene removal is not restricted to the process variant described here. Alternative isolation methods are described in the article in Ullmann cited at the start.

One development of the invention provides for some of the product mixture to be returned and mixed with the oligomerizate or the fraction rich in 1-butene or in the isomerizate. In this way, materials of value that are thus far unconverted can be subjected again to the isomerization and/or the ODH. What is recycled is a quantitative portion of the product mixture obtained from the ODH and/or a physical portion of the product mixture, for instance a residue from the butadiene removal depleted of butadiene.

Preferably, a C4-hydrocarbon stream obtained from the butadiene separation is recycled before the isomerization and/or the oxidative dehydrogenation in order to convert the butene not reacted in the first pass to butadiene.

Particularly preferably, isomerization and oxidative dehydrogenation take place at similar temperatures and pressures because in so doing it is possible to dispense with energy-intensive interim compression or decompression or heating and cooling of the isomerizate. Energy-intensive purification between the two stages is likewise not required. The oxidative dehydrogenation is preferably performed in the presence of an inert gas such as nitrogen and/or steam. A preferred embodiment of the invention envisages metered addition of steam and also of the oxygen required for the oxidative dehydrogenation after the isomerization, and accordingly feeding thereof into the stream downstream of the isomerization. In this way, the stream through the isomerization becomes smaller, which lowers the apparatus costs associated with the reactor volume.

The proportion of steam in the mixture supplied to the dehydrogenation is preferably 1 to 30 molar equivalents based on the sum total of 1-butene and 2-butene, preferably 1 to 10 molar equivalents based on the sum total of 1-butene and 2-butene. The oxygen content in the mixture supplied to the dehydrogenation is preferably 0.5 to 3 molar equivalents based on the sum total of 1-butene and 2-butene, preferably 0.8 to 2 molar equivalents based on the sum total of 1-butene and 2-butene.

The sum total of all the proportions of different substances in % by volume adds up to a total proportion of 100% by volume.

The overall process sequence will now be illustrated by reference to a process diagram:

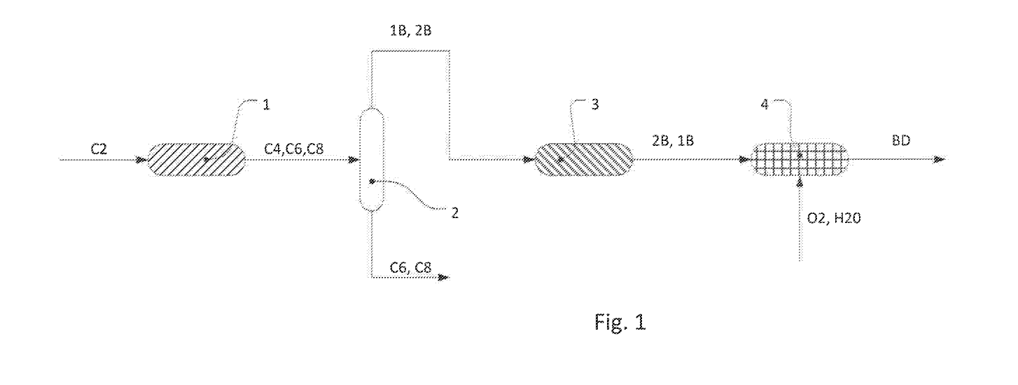

FIG. 1: Process sequence (diagrammatically)

A feed mixture C2 comprising ethene or consisting of ethene is conveyed to an oligomerization 1.

There, the ethene present in the feed mixture is dimerized to 1-butene, cis-2-butene and trans-2-butene. Moreover, higher oligomers such as C6 and C8-olefins are formed. The oligomerizate C4, C6, C8 is drawn off from the oligomerization. In a distillation 2, the oligomerizate is worked up into a 1-butene-rich fraction 1B, 2B and a fraction C6, C8 comprising the higher oligomers. These can be used elsewhere. If the ethene C2 should not be completely converted in the oligomerization 1, unreacted ethene is additionally found in the oligomerizate, and this can be separated off from the oligomerizate and be recycled to the oligomerization. This is not shown in FIG. 1.

The 1-butene-rich fraction 1B, 2B is then subjected to an isomerization 3, which shifts the thermodynamic equilibrium in the 2-butene direction, meaning that an isomerizate 2B, 1B is obtained whose content of 2-butene is significantly greater than the content of 2-butene in the oligomerizate. Nevertheless, 1-butene is still present in the isomerizate, but no longer as much as in the 1-butene-rich fraction 1B, 2B.

This isomerizate 2B, 1B enriched in 2-butene is then subjected to an oxidative dehydrogenation 4, for which purpose oxygen O2 and steam H2O is added.

This gives a product mixture BD comprising the desired 1,3-butadiene and a number of accompanying components which also have to be separated off from the product mixture. This is not shown in FIG. 1. The isolation of butadiene from the product mixture takes place as is customary in C4 technology, or by extraction.

If the oligomerizate comprises no or only a few higher oligomers, it can also be conveyed directly to the isomerization without work-up. The distillation 2 can then be dispensed with.

### LIST OF REFERENCE NUMERALS

1 oligomerization

2 work-up

3 isomerization

4 oxidative dehydrogenation

C2 feed mixture (ethene)

C4, C6, C8 oligomerizate

1B, 2B fraction rich in 1-butene

C6, C8 higher oligomers

2B, 1B isomerizate

BD product mixture (butadiene)

1. Process for the production of butadiene from ethene having the following steps:
a) providing a feed material which comprises ethene, or consists of ethene;
b) subjecting the feed material to an oligomerization to give an oligomerizate, where the oligomerizate comprises at least 1-butene;
c) optionally working-up of the oligomerizate to give a fraction rich in 1-butene;
d) subjecting the oligomerizate or the 1-butene-rich fraction to an isomerization to give an isomerizate, where the content of 2-butene in the isomerizate is greater than the content of 2-butane in the oligomerizate or the fraction rich in 1-butene;
e) subjecting the isomerizate to an oxidative dehydrogenation to give a product mixture comprising butadiene.

2. Process according to claim 1, having the following steps:
a) providing a feed material which comprises ethene or consists of ethene;
b) subjecting the feed material to an oligomerization to give an oligomerizate, where the oligomerizate comprises at least one 1-butene;
c) subjecting the oligomerizate to an isomerization to give an isomerizate, where the content of 2-butene in the isomerizate is greater than the content of 2-butene in the oligomerizate;
d) subjecting the isomerizate to an oxidative dehydrogenation to give a product mixture comprising butadiene.

3. Process according to claim 1, having the following steps:
a) providing a feed material which comprises ethene or consists of ethene;
b) subjecting the feed material to an oligomerization to give an oligomerizate, where the oligomerizate comprises at least 1-butene;
c) working-up of the oligomerizate to give a fraction rich in 1-butene;
d) subjecting the 1-butene-rich fraction to an isomerization to give an isomerizate, where the content of 2-butene of the isomerizate is greater than the content of 2-butene of the fraction rich in 1-butene;
e) subjecting the isomerizate to an oxidative dehydrogenation to give a product mixture comprising butadiene.

4. Process according to claim 1, characterized in that the oligomerization is heterogeneously catalysed.

5. Process according to claim 1, characterized in that the oligomerization is homogeneously catalysed.

6. Process according to claim 1, characterized in that the oxidative dehydrogenation takes place in the presence of a heterogeneous catalyst which comprises bismuth and molybdenum.

6. according to claim 6, characterized in that the catalyst in whose presence the oxidative dehydrogenation takes place is present in powder form.

8. Process according to claim 1, characterized in that 1-butene is converted to 2-butene in the course of the isomerization.

9. Process according to claim 1, characterized in that the isomerization takes place over a heterogeneous isomerization catalyst which comprises at least two different components, where the two components are mixed together or where the first component is applied to the second component, the first component being an alkaline earth metal oxide which is in particular selected from the group comprising magnesium oxide, calcium oxide, strontium oxide, barium oxide, and where the weight fraction of the alkaline earth metal oxide in the total isomerization catalyst is between 0.5 and 20%.

10. Process according to claim 1, characterized in that it additionally comprises a step in which butadiene is isolated from the product mixture.
